(19) United States
(12) Patent Application Publication
(10) Pub. No.: US 2015/0013689 A1
(43) Pub. Date: Jan. 15, 2015

(54) ANATOMICAL ORIENTATION SYSTEM

(71) Applicant: Howard L. Shackelford, Wheeling, WV (US)

(72) Inventor: Howard L. Shackelford, Wheeling, WV (US)

(21) Appl. No.: 14/367,164

(22) PCT Filed: Dec. 19, 2011
(86) PCT No.: PCT/US11/65825

§ 371 Date: Jun. 19, 2014

(51) Int. Cl. A61B 19/08; A61B 8/00; A61B 5/00; A61B 6/00; A61B 5/055

(52) U.S. Cl. CPC A61B 19/088; A61B 6/00; A61B 5/055; A61B 5/0073; A61B 8/00; A61B 2019/085
USPC 128/853; 128/849

(57) ABSTRACT

An anatomical orientation system comprising one or both of a physical surgical drape or a virtual surgical drape is disclosed. Physical and virtual drapes of the provided system comprise at least one anatomical diagram correlating with the anatomy of at least a portion of a patient's body requiring a surgical procedure. A drape of the provided system is adapted to be placed over, or projected onto, at least a portion of a patient's body where a surgical procedure will be performed.

### FIELD

This application relates to surgical drapes and an anatomical orientation system comprising the same. The provided system includes physical drapes, virtual drapes, or both, that comprise at least one anatomical diagram correlating with the anatomy of at least a portion of a patient's body requiring a surgical procedure.

### BACKGROUND

Surgical drapes are traditionally used to create and maintain an effective barrier against microorganisms when performing a surgical procedure. Drapes typically have materials selected to be resistant to blood and other fluids, as well as abrasion. Although many types of drapes are known in the art, there nevertheless remains a need for improved drapes.

Similar to the ongoing need for improved drapes, there is ongoing need for ways to improve how surgical procedures are performed. In any surgical procedure, problems may arise when there exists a distortion of the surgical site to an extent that the patient's anatomy is difficult to visualize. Distortion of patient anatomy may be due to wear, age, bodily dimension, disease, trauma, blood, scar tissue, or be due to otherwise abnormal or obscured anatomy. Problems visualizing patient anatomy may be magnified if the surgical procedure is to be performed in less than ideal locations (for example, trauma scenes or battle fields) or by an inexperienced clinician. Moreover, if a patient is uncooperative, difficulty in visualizing patient anatomy may occur.

As one of skill in the art will appreciate, problems visualizing patient anatomy may give rise to a clinician beginning a surgical procedure at an incorrect site or give rise to difficulties during a procedure. For example, insertion of a central venous catheter (“central line”) into a patient's internal jugular vein, external jugular vein, subclavian vein, femoral vein, or other vein requires correct identification of where to insert the catheter into the patient's body so that it is in the correct vein. This requires an accurate visualization of the vein in the body for proper placement of the catheter, and if there is distortion of patient anatomy, complications can arise. As another example, performing an appendectomy in minimal time and with minimal scarring requires knowing the exact location of the appendix in the body, which may be difficult since an appendix can lie low in the pelvis or be high under the liver. In light of the aforementioned, there exists within the art an ongoing need for improved ways of visualizing patient anatomy when performing surgical procedures.

There additionally exists within the art a need for improved systems and methods for teaching how to perform surgical procedures that require accurate visualization of patient anatomy. For example, improved systems for teaching a medical student how to insert a central line are needed. Increasingly, medical students learn anatomy with synthetic organs and online anatomic courses. While valuable, such educational tools lack the context of a human body. Thus, improved teaching systems and methods for visualizing patient anatomy in context can be useful in the education of medical professionals.

### SUMMARY

It is an object of the present application to meet these various needs in the art by disclosing an anatomical orientation system. In some embodiments, the provided system may be used to aid a clinician in the identification of a surgical site, to aid a clinician in the performance of surgical procedure, to aid a clinician in learning how to perform a surgical procedure, or combinations thereof. In various embodiments, the provided system comprises one or both of a physical surgical drape or a virtual drape. Physical and virtual drapes of the provided system comprise at least one anatomical diagram correlating with the anatomy of at least a portion of a patient's body requiring a surgical procedure. A physical drape additionally comprises an incise material and is adapted to be physically placed over at least a portion of a patient's body requiring a surgical procedure. A virtual drape is adapted to be projected onto at least a portion of a patient's body requiring a surgical procedure.

When a drape of the provided system is placed over, or projected onto, at least a portion of a patient's body where the surgical procedure will be performed, the system enables a clinician to accurately identify the proper location for performing a surgical procedure. It also enables a clinician to use the diagram as an anatomical reference or guide during a surgical procedure.

In some embodiments, provided are methods of performing one or more surgical procedures, wherein said methods comprise placing, or projecting, the drape of the provided system over at least a portion of a patient's body where the surgical procedure will be performed.

In some embodiments, provided are kits for use in performing one or more surgical procedures, wherein said kits comprise a physical drape, a virtual drape, or both. Thus, the provided kits comprise an anatomical diagram correlating with the anatomy of at least a portion of a patient's body requiring a surgical procedure. In some embodiments, the provided kits may also comprise a surgical drape comprising an incise material.

These and additional embodiments of the present application will become apparent in the course of the following description.

### BRIEF DESCRIPTION OF THE DRAWINGS

A more complete appreciation of the various embodiments of the present application will be readily obtained as the same becomes better understood by reference to the following detailed description when considered in connection with the accompanying drawings, wherein:

FIG. 1 illustrates one example of an anatomical diagram correlating with the anatomy of at least a portion of a patient's body, wherein such diagram may be one component of an anatomical orientation system;

FIG. 2 illustrates another example of an anatomical diagram correlating with the anatomy of at least one portion of a patient's body, wherein such diagram may be one component of an anatomical orientation system;

FIG. 3 illustrates yet another example of an anatomical diagram correlating with the anatomy of at least one portion of a patient's body, wherein such diagram may be one component of an anatomical orientation system;

FIG. 4 is one example of an anatomical orientation system comprising a physical drape.

### DESCRIPTION OF EMBODIMENTS

Specific embodiments of the present application will now be described. The invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the specification and appended claims, the singular forms “a,” “an,” and “the” are intended to include the plural forms as well, unless the context clearly indicates otherwise.

As used in the specification and appended claims, the term “clinician” is intended to mean a medical doctor, a medical student, a nurse, a nursing student, an emergency medical technician (EMT), an EMT student, or any person otherwise authorized to perform a medical procedure.

The term “medical procedure,” as used in the specification and appended claims, is intended to mean any procedure involving puncture or incision of the skin.

Unless otherwise indicated, all numbers expressing quantities of ingredients, properties such as molecular weight, reaction conditions, and so forth as used in the specification and claims are to be understood as being modified in all instances by the term “about.” Additionally, the disclosure of any ranges in the specification and claims are to be understood as including the range itself and also anything subsumed therein, as well as endpoints. Unless otherwise indicated, the numerical properties set forth in the specification and claims are approximations that may vary depending on the desired properties sought to be obtained in embodiments of the present invention. Notwithstanding that numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

In various embodiments, provided is an anatomical orientation system that may be used in the performance of one or more surgical procedures to improve visualization of patient anatomy. The system comprises an anatomical diagram (literal image, non-literal image, or combination thereof) of at least a portion of a patient's body, said system adapted to orient a clinician to one or more suitable places on a patient's body for performing one or more surgical procedures. When the system is used, the anatomical diagram may be referenced as needed by a clinician to aid in performance of a surgical procedure by providing accurate visualization of patient anatomy. The system may also be used for instructing a clinician in how to perform a surgical procedure requiring accurate visualization of patient anatomy.

The provided system comprises a drape. In some embodiments, the drape is a physical drape. In some embodiments, the drape is a virtual drape. In some embodiments, the system comprises both a physical and a virtual drape. When properly placed or projected, the drape provides a clinician with an accurate visualization of the underlying anatomy of the patient. Thus, whether comprising a physical drape, a virtual drape, or both, the provided system enables a clinician to accurately identify the proper location for performing a surgical procedure, as well as a reference or guide to the anatomy during performance of the procedure. In some embodiments, it is contemplated that the system allows for performing more than one surgical procedure using the same drape. A surgical procedure performed using the provided system may be selected from a puncture (such as insertion of a needle or central line) or an incision (such as by scalpel). For example, the surgical procedure may be insertion of a central line into a patient's internal jugular vein, external jugular vein, subclavian vein, femoral vein, or other vein.

### Physical Drape

In various embodiments, the provided system comprises a physical drape. A physical drape of the provided system is adapted to be placed over at least a portion of a patient's body where a surgical procedure will be performed. In various embodiments, a physical drape comprises an incise material that is suitably flexible to be formable to a contour of a patient's anatomy without substantially deforming the anatomy. Typically, an incise material is a sterilizable polymeric film, mesh, or combination thereof, having an adhesive on at least a portion of a surface that is adapted to contact the patient's skin (“Adhering Surface”). Any incise material suitable for medical use may be used with the provided system. In an exemplary embodiment, the incise material may be a film (for example, low-density polyethylene film) that is clear, translucent, or opaque, or a combination thereof. The incise material may be hydrophobic. The incise material may be porous or non-porous. The incise material may be treated with or impregnated with antibiotics, silver-containing substances (for example, silver sulfadiazine), chlorhexidine, other anti-microbial agents, or combinations thereof. Incise materials may be obtained from, for example, 3M; Bertek, Inc.; Medical Concepts Development; and T. J. Smith and Nephew Ltd. In some embodiments, the incise material comprises at least one opening or aperture (“Fenestration”) through which the surgical procedure is performed. In some embodiments, the incise material does not have a Fenestration, and the surgical procedure is performed directly through the incise material.

The adhesive of the incise material is at least partially covered with a removable backing until readied for use. In practice, the removable backing is removed and the Adhering Surface of the material is applied directly to the skin of the patient in a manner that conforms with patient anatomy without distorting patient anatomy. The removable backing may be of any material suitable for medical use. Examples include, but are not limited to, wax-coated papers and silicone-coated papers. The adhesive may be applied to all of the Adhering Surface of the incise material. Alternatively, the adhesive may be applied to only a portion of the Adhering Surface of the incise material. For example, adhesive may be applied to the periphery of the Adhering Surface. As another example, adhesive may be applied to the periphery of a Fenestration in the incise material. Any adhesive suitable for medical use may be used with the provided system. One example of a medically suitable adhesive is Integuseal® (Kimberly Clark).

In various embodiments of the system, the incise material is adapted to comprise, or adapted to receive, a diagram of at least a partial anatomy of the area of the patient's body to be covered by the drape. The diagram may be a photograph or other literal anatomical image (such as x-ray, MRI, ultrasound, or tomography-generated image), or may be a non-literal anatomical image (such as a drawing, illustration, sketch, or graphic), or may be a combination thereof. The diagram may additionally comprise instructions, patient identifying information, or other relevant textual material. In various embodiments, the diagram comprises trackable landmarks to aid in proper positioning of the drape. The diagram may be incorporated into or onto the incise material by any suitable technique. It is contemplated that the diagram may be laminated, adhered, taped, printed, sewed, or otherwise formed or secured on or in the drape. For example, it is contemplated that the diagram may be applied to the incise material by any suitable printing technique, including but not limited to, manual drawing, offset lithography, flexography, dye transfer, inkjet printing, laser printing, pad printing, relief printing, rotogravure printing, and screen-printing. It is also contemplated that the diagram may be a label (such as a sticker or decal) that is applied to the incise material and is adhered thereto by permanent adhesive, peelable adhesive, ultra-peelable adhesive, high tack adhesive, or by static cling. In some embodiments, the diagram may be a removable label that may be removed from the incise material after performing at least one surgical procedure, thereby leaving the underlying incise material adhered to the patient. For example, it is contemplated that after placement of a central line using the system, the diagram may be removed, thereby leaving the underlying incise material in place for at least one additional surgical procedure. It is additionally contemplated that the diagram may be applied to a first layer of incise material, and a second layer of incise material is applied thereto, thereby forming a drape comprising a first incise layer, a second incise layer, and a diagram disposed between the first and second incise layers. Regardless of how applied to, adhered to, or received by the incise material, the diagram may be transparent, translucent, opaque, or a combination thereof.

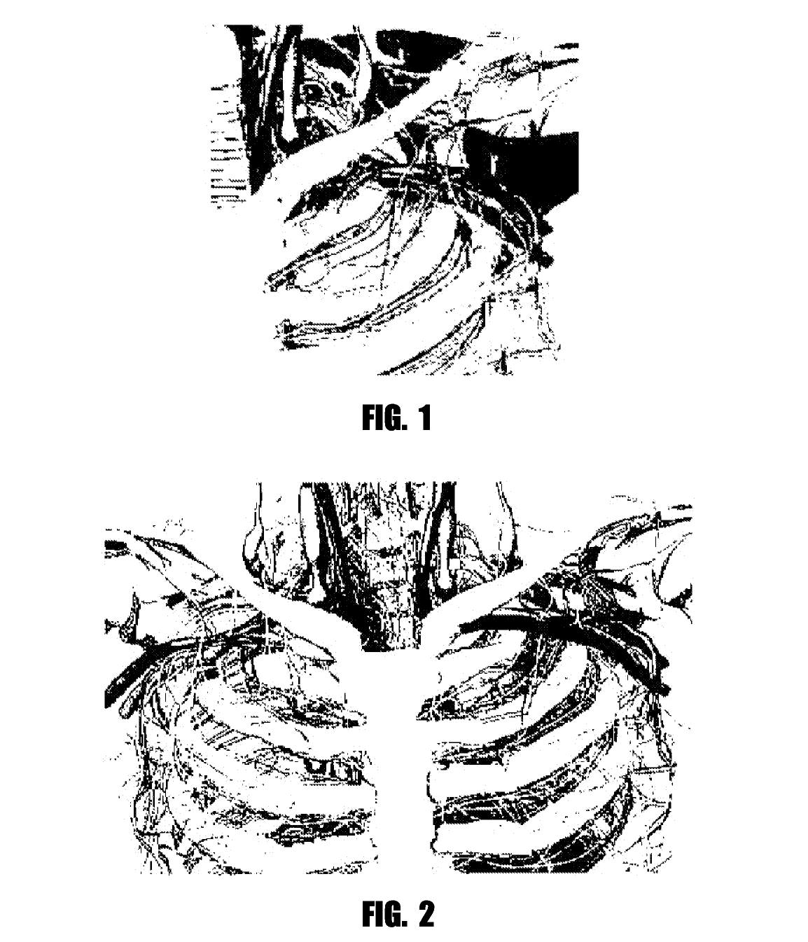

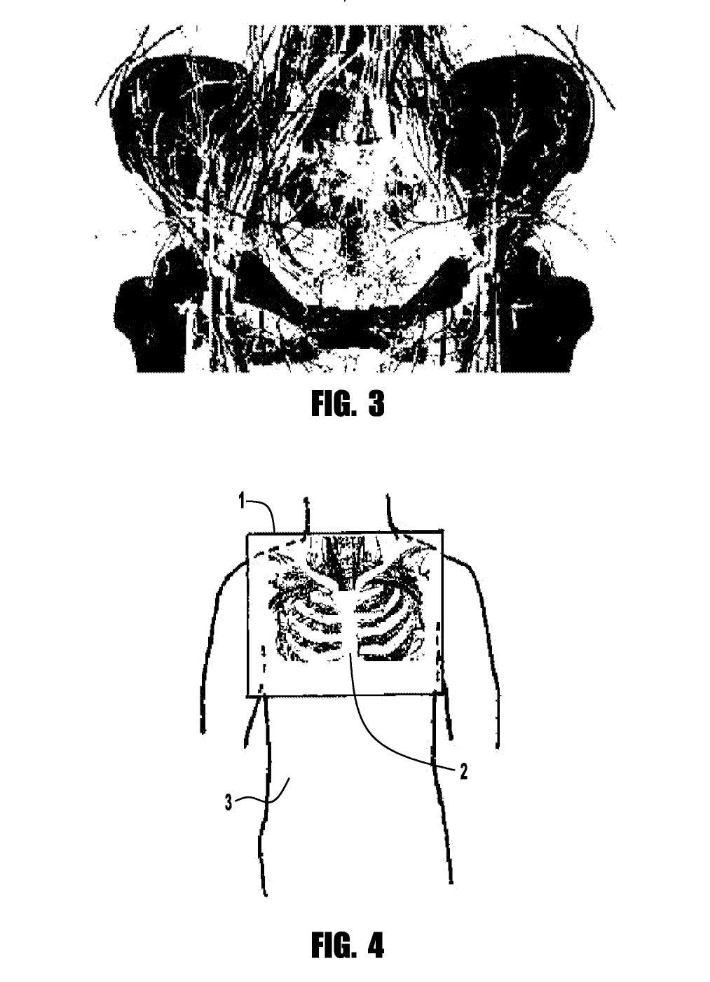

Non-limiting examples of anatomical diagrams suitable for use with the provided physical drape include those depicted in FIGS. 1-3. Referring to FIG. 1, shown is an anatomical diagram of the left clavicle with localization of surrounding anatomy, including subclavian and internal jugular veins. The clavicle shown therein may be used as a landmark to aid in proper placement of the physical drape. It is contemplated that a physical drape comprising such diagram may be used to guide needle placement for central lines. Referring to FIG. 2, shown is an anatomical diagram of bilateral vascular structures. It is contemplated that a physical drape comprising such diagram may be used as a guide for central line access. Referring to FIG. 3, shown is an anatomical diagram of pelvic and femoral vascular structures. It is contemplated that a physical drape comprising such diagram may be used as an access guide to one or both of the femoral artery and vein. Although several examples are provided herein, one of skill in the art will recognize that said examples are for illustrative purposes only and are not intended to be limiting.

In some embodiments of the system, a physical drape may comprise an absorbent material. Any absorbent material suitable for medical use may be used. For example, it is contemplated that the absorbent material may be selected from spun-bond fabrics, melt-blown fabrics, and paper. If the system comprises an absorbent material, the incise material is adapted to comprise or receive the absorbent material. The absorbent material may be adhered to the incise material or otherwise attached to or incorporated with it. For example, the drape may comprise a layer of absorbent material attached to at least a portion of the incise material, such as the surface of the incise material opposite to the Adhering Surface. As another example, the drape may comprise absorbent material attached to at least a portion of the periphery of the incise material.

Referring to FIG. 4, depicted is one example of a provided anatomical orientation system comprising a physical drape 1. The drape 1 is a barrier surgical drape comprising an anatomical diagram 2, and is adapted to be applied to an area of the patient's body 3 having corresponding anatomy. Proper orientation and placement may be obtained by matching landmarks (eg., clavicle) on both the diagram 2 and the patient 3.

### Virtual Drape

In various embodiments, the provided system comprises a virtual drape. A virtual drape of the provided system is adapted to be projected onto at least a portion of a patient's body where a surgical procedure will be performed, onto a conventional or provided physical drape covering at least a portion of a patient's body where a surgical procedure will be performed, or both. In practice, the portion of a patient's body where a surgical procedure will be performed is located, and a projector is focused to project the virtual drape onto the located portion of the patient's body (or a physical drape covering said located portion). Often the projector is set up with its optical axis perpendicular to the portion of the located portion of the patient body (or a physical drape covering said located portion), and the size of the projected virtual drape is customized to fit the patient by the adjusting the focal length. However, one of skill in the art will understand that such a set up may be varied without departing from the scope of the present application.

Any projector suitable for use in a medical setting may be used with the provided system, including stationary projectors, portable projectors, and pocket projectors. Some projectors may be mounted on a ceiling, wall, boom, or cart. In some embodiments, 3D projectors may be used. Illustrative examples of suitable projectors are Canon® REALiS SX80 Mark II D Projector, Mitsubishi® MH2850 SXGA LCD Medical-Projector, DepthQ® HDs3D-1 Stereoscopic 3D Projector, and Optoma® PK102 pocket projector.

In various embodiments of a system comprising a virtual drape, the system is adapted to project a diagram of at least a partial anatomy of the area of the patient's body to be covered by the drape. The diagram may be a photograph or other literal anatomical image (such as x-ray, MRI, ultrasound, or tomography-generated image), or may be a non-literal anatomical image (such as a drawing, illustration, sketch, or graphic), or may be a combination thereof. The diagram may additionally comprise instructions, patient identifying information, or other relevant textual material. In various embodiments, the diagram comprises trackable landmarks to aid in proper positioning, sizing, or both of the projected diagram. As one illustrative example of projectable diagrams, the virtual drape may comprise a MRI scan of the specific patient on which a medical procedure will be performed. As another illustrative example, the projected diagram may be a CAT scan of the specific patient on which a medical procedure will be performed. As one illustrative example of application of the system, a CAT scan of the appendix of a patient with appendicitis may be obtained, and using the provided system, a diagram of the appendix may be projected onto the patient prior to and during an appendectomy procedure, thereby enabling a clinician in knowing exactly where the patient's appendix is located. This is advantageous because the location of an appendix is not fixed; it can lie low in the pelvis or it can be high under the liver. Other contemplated examples of application are to project a diagram of an adrenal mass or a pancreatic mass into the field of surgery.

Non-limiting examples of anatomical diagrams suitable for use with the provided virtual drape include those depicted in FIGS. 1-3. Referring to FIG. 1, shown is an anatomical diagram of the left clavicle with localization of surrounding anatomy, including subclavian and internal jugular veins. The clavicle shown therein may be used as a landmark to aid in proper placement of the physical drape. It is contemplated that a virtual drape comprising such a projectable diagram may be used to guide needle placement for central lines. Referring to FIG. 2, shown is an anatomical diagram of bilateral vascular structures. It is contemplated that a virtual drape comprising such a projectable diagram may be used as a guide for central line access. Referring to FIG. 3, shown is an anatomical diagram of pelvic and femoral vascular structures. It is contemplated that a virtual drape comprising such a projectable diagram may be used as an access guide to one or both of the femoral artery and vein. Although several examples are provided herein, one of skill in the art will recognize that said examples are for illustrative purposes only and are not intended to be limiting.

A virtual drape has the advantage of overlaying anatomy that is of interest without regard to compromising sterility of the field. Thus, in some embodiments, the provided system allows for visualization of patient anatomy without the use of a physical drape to cover said field. However, it may be desirable or necessary that the field be covered with a physical drape. Thus, in some embodiments, the provided system allows for projecting a virtual drape onto at least a portion of a conventional physical drape overlaying the anatomy of interest. For example, a virtual drape may be projected onto a clear incise material. In some embodiments, the provided system allows for projecting a virtual drape onto at least a portion of a provided physical drape overlaying the anatomy of interest. For example, a virtual drape may be projected onto at least a portion of a provided drape.

### Kits

The provided drapes may be manufactured and sold as separate drapes, or as part of a surgical pack. Accordingly, in various embodiments, provided is a kit for draping a surgical site comprising the provided one or more of the provided drapes and instructions, wherein the drape is positioned over (and/or projected onto) the surgical site on the patient's body, and when so positioned or projected, a clinician may visualize anatomy of the underlying patient body when performing a surgical procedure. In some embodiments, the anatomy visualized is specific to the individual patient. For example, the diagram may be derived from an x-ray, MRI, tomography scan, ultrasound, or other patient-specific image. In some embodiments, the anatomy visualized is generalized for the patient (for example, an illustration). However, it is contemplated that in regards to such embodiment, the provided drape may be manufactured and sold in a plurality of variations of generalized anatomy, thereby allowing a drape for use on the patient to be selected based upon one or more determinable factors specific to the patient. Examples of such factors include, but are not limited to, site condition, site location, patient age, patient gender, body size, body weight, and other such determinable factors.

The instructions of the kit provide guidance on the selection and sequence of use of the items in the kit. If sold as a kit for a specific surgical procedure, the instructions may also provide guidance as to use of the drape in the context of the surgical procedure. For example, a kit for insertion of a central line may provide guidance on the selection and sequence of use of the items in the kit, as well as instructions for using the drape to orient the clinician to proper insertion and placement of the central line. In some embodiments, it is contemplated that at least some instructions may be applied to the drape in a manner consistent with how a diagram may be applied to a drape, as described herein.

### Methods

In various embodiments, provided are methods of using the provided system to identify a surgical site on a patient prior to performance of a surgical procedure. Also provided are methods of using the provided system to aid a clinician in performance of a surgical procedure by providing an anatomical diagram that can be referenced as needed during the procedure. Accordingly, in some embodiments, provided are methods for correlating a selected portion of a patient's body to an anatomical diagram for performance of a surgical procedure, said methods comprising: (i) applying to a selected portion of a patient's body a provided physical drape comprising an incise material and a diagram depicting anatomy corresponding to at least a portion of the selected portion of the body; (ii) placing the physical drape over, adhering the physical to, or both, at least a portion of the selected portion of the body such that the depicted anatomy and the selected portion of the body anatomically correlate; (iii) locating a surgical site on the selected portion of the body by referencing the depicted anatomy on the drape; and (iv) performing the surgical procedure through the drape, wherein the depicted anatomy may be referenced as needed during the procedure.

In practice, the surgical area of the patient is first scrubbed and treated with an anti-microbial agent. If the physical drape used comprises and Adhering Surface, removable backings covering the adhesive of the Adhering Surface of the incise material are removed, and the Adhering Surface of the incise material is applied directly to the skin of the patient over at least a portion of the body where the surgical site is located. Application of the drape to the patient's body is done in a manner allowing for correlation between the anatomy depicted in the diagram and the anatomy of the patient. The drape is adapted to be flexible and conform to the contours of the patient's anatomy, thereby aiding in correlating the depicted anatomy with the patient's actual anatomy. Once properly adhered or otherwise placed onto the patient, a clinician may use the depicted anatomy to locate a surgical site on the body. If the depicted anatomy is generalized anatomy selected from a plurality of available diagrams using determinable patient data (eg., patient age, patient gender, patient dimensions, or combinations thereof), the surgical site may be located with an acceptable level of accuracy. If the depicted anatomy is the patient's individual anatomy (eg., x-ray), the surgical site may be located with the same or better level of accuracy. Once the surgical site is located, the surgical procedure may be performed through a fenestration in the drape. Alternatively, the procedure may be performed by directly performing a puncture or incision through the drape. During the procedure, a clinician may refer to the depicted anatomy as needed to guide the procedure or portion thereof. For example, if the surgical procedure is insertion of a central line, the clinician may refer to the depicted anatomy as needed when first inserting the catheter into the relevant vein, as well as to aid in determining correct placement of the catheter. Upon conclusion of the procedure, or at any time before then, the clinician may remove the drape. If the diagram of the drape is removable, the clinician may elect to remove the diagram at any time during the procedure or upon completion, thereby leaving the remainder of the drape. If more than one surgical procedure is to be performed, this may be desirable.

In some embodiments, the provided system comprises a virtual drape. Accordingly, in some embodiments, provided are methods for correlating a selected portion of a patient's body to an anatomical diagram for performance of a surgical procedure, said methods comprising: (i) projecting onto a selected portion of a patient's body, a virtual drape comprising a diagram depicting anatomy corresponding to at least a portion of the selected portion of the body; (ii) adjusting the focus, size, location, or combination thereof of the projected diagram such that the depicted anatomy and the selected portion of the body anatomically correlate; (iii) locating a surgical site on the selected portion of the patient body by referencing the depicted anatomy on projected diagram; and (iv) performing the surgical procedure through the projected diagram, wherein the depicted anatomy may be referenced as needed during the procedure.

The present invention should not be considered limited to the specific examples described herein, but rather should be understood to cover all aspects of the invention. Various modifications, equivalent processes, as well as numerous structures and devices to which the present invention may be applicable will be readily apparent to those of skill in the art. Those skilled in the art will understand that various changes may be made without departing from the scope of the invention, which is not to be considered limited to what is described in the specification.

### EXAMPLES

### Example 1

### Contemplated Use of a Physical Drape

The surgical area of the patient is prepared and dried in accordance with standard surgical procedures with which one of skill in the art would be familiar. Thereafter, a barrier surgical drape comprising an anatomical diagram is applied to an area of the patient's body having corresponding anatomy, and landmarks (eg., clavicle or pelvice anterior superior spine) on both the diagram and the patient are matched in order to obtain proper placement of the drape. The anatomy depicted on the drape is used by the clinician as a proxy for the underlying patient anatomy, thereby guiding the clinician to the appropriate anatomy for the desired surgical procedure. For example, vasculature depicted on the drape may be used as a guide for cannulation. Once the surgical procedure has been completed, the drape is removed.

### Example 2

### Contemplated Use of a Virtual Drape

The surgical area of the patient is prepared and dried in accordance with standard surgical procedures with which one of skill in the art would be familiar. Thereafter, a virtual drape comprising an anatomical diagram is projected (using a projector, such as Optoma® PK102 pocket projector) onto an area of the patient's body having corresponding anatomy, and landmarks (eg., clavicle or pelvice anterior superior spine) on both the diagram and the patient are matched in order to obtain proper orientation of the drape. Additionally, the projected diagram is size adjusted to the landmarks. The projected anatomy is used by the clinician as a proxy for the underlying patient anatomy, thereby guiding the clinician to the appropriate anatomy for the desired surgical procedure. For example, vasculature of a projected diagram may be used as a guide for cannulation. Once the surgical procedure has been completed, the projector is turned off. One of skill in the art will appreciate that the virtual drape may be used in combination with a standard surgical drape, such as a Ioban™ 2 antimicrobial incise drape (3M).

What is claimed is:

1. An anatomical orientation system for use in the performance of one or more surgical procedures on a subject, comprising:
a drape selected from one or more of a physical drape and a virtual drape;
an anatomical diagram of at least a portion of the subject's body, the diagram selected from a literal anatomical image, a non-literal anatomical image, or combination thereof;
wherein the drape is adapted to be adjustable such that the anatomical diagram and the portion of the subject's body anatomically correlate;
wherein the system is adapted to orient a clinician to one or more surgical sites on the subject's body, to guide a clinician in the performance of a surgical procedure on the subject's body, or combinations thereof.

2. An anatomical orientation system according to claim 1, wherein the drape is a physical drape adapted to be placed over at least a portion of a subject's body where a surgical procedure will be performed.

3. An anatomical orientation system according to claim 2, wherein the drape comprises at least one layer of incise material adapted to comprise or receive the anatomical diagram.

4. An anatomical orientation system according to claim 3, wherein the anatomical diagram is selected from an x-ray, MRI, ultrasound, tomography-generated image, drawing, illustration, sketch, or graphic.

5. An anatomical orientation system according to claim 4, wherein the anatomical diagram comprises text, trackable landmarks, or combinations thereof.

6. An anatomical orientation system according to claim 3, wherein the diagram is transparent, translucent, opaque, or a combination thereof.

7. An anatomical orientation system according to claim 3, wherein the diagram is laminated, adhered, taped, printed, or sewed onto or into the incise material.

8. An anatomical orientation system according to claim 3, wherein the diagram is removable.

9. An anatomical orientation system according to claim 3, comprising more than one layer of incise material.

10. An anatomical orientation system according to claim 9, wherein the diagram is received between two layers of incise material.

11. An anatomical orientation system according to claim 3, wherein the incise material is a sterilizable polymeric film, mesh, or combination thereof, having at least one surface adapted to contact the subject's skin, and wherein the incise material comprises an adhesive on at least a portion of said surface.

12. An anatomical orientation system according to claim 11, wherein the incise material is coated or impregnated with one or more anti-microbial agents.

13. An anatomical orientation system according to claim 3, wherein the incise material comprises at least one fenestration through which the surgical procedure may be performed.

14. An anatomical orientation system according to claim 3, wherein drape further comprises an absorbent material.

15. An anatomical orientation system according to claim 1, wherein the drape is a virtual drape adapted to be projected onto at least a portion of a subject's body where a surgical procedure will be performed, onto a physical drape covering at least a portion of a subject's body where a surgical procedure will be performed, or both.

16. An anatomical orientation system according to claim 15, wherein the anatomical diagram is selected from an x-ray, MRI, ultrasound, tomography-generated image, drawing, illustration, sketch, or graphic.

17. An anatomical orientation system according to claim 16, wherein the anatomical diagram comprises text, trackable landmarks, or combinations thereof.

18. An anatomical orientation system according to claim 16, wherein the diagram is transparent, translucent, opaque, or a combination thereof.

19. A method of identifying a surgical site on a subject in need of a surgical procedure, comprising:
(i) applying to a pre-determined portion of a subject's body an anatomical orientation system, comprising: (a) a drape selected from one or more of a physical drape and a virtual drape; and (b) an anatomical diagram of at least a portion of the pre-determined portion of a subject's body, the diagram selected from a literal anatomical image, a non-literal anatomical image, or combination thereof;
(ii) adjusting the drape such that the anatomical diagram and the pre-determined portion of the subject's body anatomically correlate; and
(iii) locating, prior to performing the surgical procedure through the drape, a surgical site on the pre-determined portion of the subject's body by referencing the anatomy depicted on the adjusted drape.

20. A surgical drape, comprising:
(i) an anatomical diagram of at least a portion of a subject's body on which a surgical procedure will be performed, the diagram selected from an x-ray, MRI, ultrasound, tomography-generated image, drawing, illustration, sketch, graphic, or combination thereof;
wherein the anatomical diagram optionally comprises text, trackable landmarks, or combinations thereof;
(ii) at least one layer of incise material adapted to comprise or receive the anatomical diagram;
wherein the incise material optionally comprises at least one fenestration through which a surgical procedure may be performed; and
wherein the drape is adapted to orient a clinician to one or more surgical sites on the subject's body, to guide a clinician in the performance of a surgical procedure on the subject's body, or combinations thereof.
